(19) United States
(12) Patent Application Publication
(10) Pub. No.: US 2026/0224553 A1
(43) Pub. Date: Aug. 6, 2026

(54) OXIME REACTIVATORS

(71) Applicant: Southwest Research Institute, San Antonio, TX (US)

(72) Inventors: Shawn T. BLUMBERG, San Antonio, TX (US); Paul W. MIGUEL, San Antonio, TX (US); Christopher L. DORSEY, San Marcos, TX (US); Ronald DOUGLAS, San Antonio, TX (US)

(21) Appl. No.: 19/044,675

(22) Filed: Feb. 4, 2025

(51) Int. Cl. A61K 31/47; C07D 215/38

(52) U.S. Cl. CPC A61K 31/47; C07D 215/38

(57) ABSTRACT

The preparation of oxime reactivators to that may be used to treat poisoning from organophosphorus (OP) compounds present in nerve agents and pesticides.

### STATEMENT REGARDING FEDERALLY SPONSORED RESEARCH OR DEVELOPMENT

This invention was made with government support under Contract No. OTA-WISQKN-16-9-1002 awarded by the Department of Defense. The government has certain rights in the invention.

### FIELD

The present disclosure is directed to the preparation of oxime reactivators to that may be used to treat poisoning from organophosphorus (OP) compounds present in nerve agents and pesticides.

### BACKGROUND

Organophosphorus nerve agents (OPNA), used as chemical weapons and pesticides cause an estimated 300,000 deaths per year worldwide and represent a significant threat to deployed military personnel. Mild exposure typically results in blurry vision, eye ache and runny nose which progresses to nausea, vomiting, and abdominal cramps upon moderate exposure and finally arrhythmias, loss of consciousness and death upon severe exposure.

Oxime compounds have been shown to reactivate acetylcholinesterase (AChE), restoring normal enzyme function. Current Food and Drug Administration (FDA) approved antidotes utilize separate solutions of 2 mL (300 mg/mL) 2-Pyridine Aldoxime Methyl Chloride (2-PAM), a reactivator, and 0.7 mL (2.99 mg/mL) Atropine (ATR), either contained in separate cartridges (MARK 1) or in 2 compartments, in a stacked arrangement for sequential delivery (ATNAA, Meridian/Pfizer). Other promising oximes that reportedly offer improved efficacy are the bis-pyridinium oximes HLö-7 dimethylsulfate (DMS), HI-6 DMS and obidoxime DMS and MMB-4. See, Wilhelm, C. M.; Snider, T, H.; Babin, M. C.; Jett, D. A.; Platoff Jr., G. E.; Yeung, D. T. “A Comprehensive Evaluation of the Efficacy of Leading Oxime Therapies in Guinea Pigs Exposed to Organophosphorus Chemical Warfare Agents or Pesticides” Toxicol. Appl. Pharm. 2014, 281, 254-265. See also, U.S. Pat. No. 8,404,850, Bis-Quaternary Pyridinium-Aldoxime Salts And Treatment of Exposure To Cholinesterase Inhibitors.

However, due to the charged nature of the pyridinium oximes, they do not penetrate into the central nervous system (CNS) and thus fail to ameliorate any of the CNS symptoms of OPNA exposure. This is a significant shortcoming and represents a substantial unmet medical need to both the warfighter and civilian populations.

### SUMMARY

A method for producing an oxime reactivator comprising:
(a) reacting 3,5-dinitro-2 (1H)-pyridone with 1,4-dioxaspiro[4.5]decan-8-one and forming the 3′-nitro-7′,8′-dihydro-5′H-spiro[1,3]dioxolane-2,6′-quinoline] having the following formula:

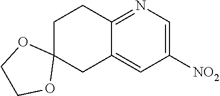 [SMILES: O=[N+]([O-])c1cnc2c(c1)CC1(CC2)OCCO1]

(b) hydrolyzing 3′-nitro-7′,8′-dihydro-5′H-spiro[1,3]dioxolane-2,6′-quinoline] and forming 3-nitro-7,8-dihydroquinolin-6 (5H)-one having the following formula:

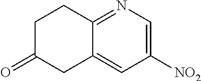 [SMILES: O=C1CCc2ncc([N+](=O)[O-])cc2C1]

(c) subjecting 3-nitro-7,8-dihydroquinolin-6 (5H)-one to reductive amination with Ar—(R₁)—NH—R₂, wherein Ar can be an aromatic or heteroaromatic ring, R₁ is an alkyl group having 2-4 carbon atoms and R₂ is a saturated or unsaturated hydrocarbon group having 1-4 carbon atoms and producing

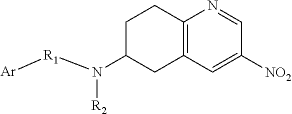

(d) reducing the nitro group on the compound formed in (c) and forming:

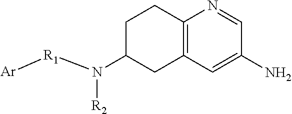

(e) coupling the compound formed in (d) with (E)-2-((allyloxy)imino)-3-oxobutanoyl chloride to form:

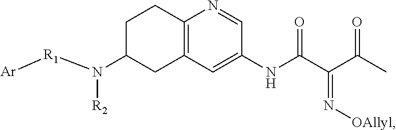

and
(f) conversion of the allyl group in the compound produced in (e) and forming:

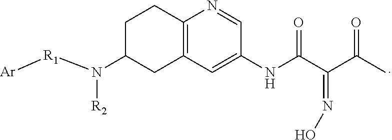

A method of reactively coupling with an acid chloride comprising:
(a) providing the following compound having the formula:

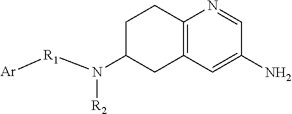

wherein Ar can be an aromatic or heteroaromatic ring, R₁ is an alkyl group having 2-4 carbon atoms and R₂ is a saturated or unsaturated hydrocarbon group having 1-4 carbon atoms;
(b) providing an acid chloride compound having the following formula wherein Allyl is reference to —CH₂—CH═CH₂:

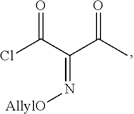

and
(c) coupling the compounds in (a) and (b) and forming:

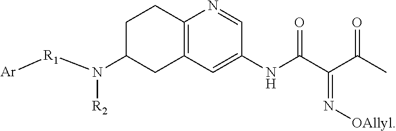

An enantiopure compound comprising the following formula:

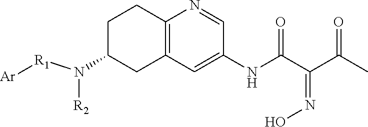

wherein Ar can be an aromatic or heteroaromatic ring, R₁ is an alkyl group having 2-4 carbon atoms, R₂ is a saturated or unsaturated hydrocarbon group having 1-4 carbon atoms and having an enantiomeric purity of at least 90%.

An enantiopure compound comprising the following formula:

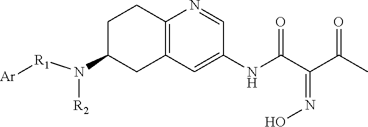

wherein Ar can be an aromatic or heteroaromatic ring, R₁ is an alkyl group having 2-4 carbon atoms, R₂ is a saturated or unsaturated hydrocarbon group having 1-4 carbon atoms and having an enantiomeric purity of at least 90%.

A therapeutic method of treating a person or animal for intoxication with a phosphorous containing cholinesterase inhibitor, comprising administering to a person or animal one or more of the following compounds:
(a) an enantiopure compound comprising the following formula:

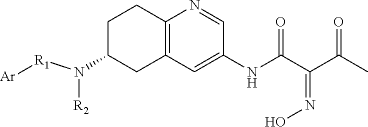

wherein Ar can be an aromatic or heteroaromatic ring, R₁ is an alkyl group having 2-4 carbon atoms, R₂ is a saturated or unsaturated hydrocarbon group having 1-4 carbon atoms and having an enantiomeric purity of at least 90%; or
(b) an enantiopure compound having the following formula:

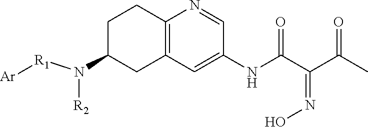

wherein Ar can be an aromatic or heteroaromatic ring, R₁ is an alkyl group having 2-4 carbon atoms, R₂ is a saturated or unsaturated hydrocarbon group having 1-4 carbon atoms and having an enantiomeric purity of at least 90%.

### DETAILED DESCRIPTION

A scalable process is now described for the preparation of oxime reactivators which may be employed for treating poisoning from organophosphorus (OP) compounds. The reactivators preferably amount to uncharged oxime reactivators which is reference to the feature that the oxime reactivator itself does not contain: (1) a quaternary substituted nitrogen, such as a nitrogen covalently bonded to four other atoms via sp3 hybridization (e.g. [NR₄]⁺ wherein in one example R₄ amounts to four C atoms bonded to the nitrogen) making it permanently positively charged regardless of pH; or (2) a nitrogen having sp2 hybridization bonded to three atoms, as in [R₁═N—R₂]⁺ wherein in one example R₁ is a carbon atom doubly bonded to nitrogen and R₂ represents two individual carbon atoms bonded to the nitrogen, again making it positively charged regardless of pH. It can therefore be appreciated that due to the charged nature of such structures, regardless of pH, they are relatively poor in their ability to cross the brain-blood barrier. Oxime itself is the general reference to an organic compound containing both a hydroxy group (—OH) and an imine bond which is understood as a carbon-nitrogen double bond as in —C═N—OH.

While pathways to more general uncharged oxime structures are now disclosed in both racemic and enantiopure forms, one particularly preferred uncharged oxime reactivator herein can also be provided as a racemate, namely (+)-2-(hydroxyimino)-N-(6-(methyl(phenethyl)amino)-5,6,7,8-tetrahydroquinolin-3-yl)-3-oxobutanamide identified as compound (+)-(9). Such compound is also now provided herein in enantiopure form such as (+)-2-(hydroxyimino)-N-(6-(methyl(phenethyl)amino)-5,6,7,8-tetrahydroquinolin-3-yl)-3-oxobutanamide identified as (9+) or (−)--2-(hydroxyimino)-N-(6-(methyl(phenethyl)amino)-5,6,7,8-tetrahydroquinolin-3-yl)-3-oxobutanamide identified as (9−), as illustrated below

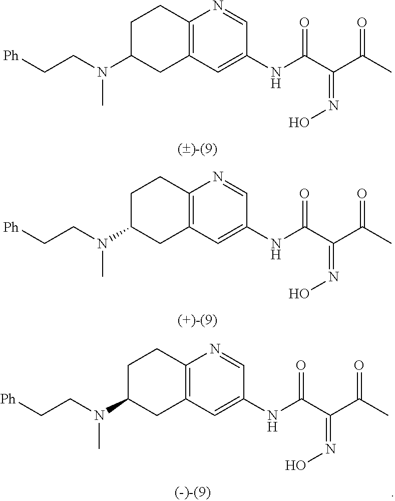 [SMILES: CC(=O)/C(=N/O)C(=O)Nc1cnc2c(c1)CC(N(C)CCc1ccccc1)CC2 ; CC(=O)/C(=N/O)C(=O)Nc1cnc2c(c1)C[C@H](N(C)CCc1ccccc1)CC2 ; CC(=O)/C(=N/O)C(=O)Nc1cnc2c(c1)C[C@@H](N(C)CCc1ccccc1)CC2]

In using the term “enantiopure” or an “enantiopure form” herein, it means that the one identified enantiomer is predominately present (90% or more). Accordingly, while relatively minor amounts of the other enantiomeric forms may be present, the desired enantiomer should constitute at least 90% of all forms of the compound. For example, enantiopure (+)-(9) or (−)-(9−) contains 90% or more of (+)-(9) or (−)-(9), and contains 10% or less of other enantiomeric forms. Preferably, the enantiopure form of (+)-(9) or (−)-(9) constitutes at least 95% of the desired enantiomer, more preferably at least 98%, and most preferably at least 99%.

A preferred and scalable route for the production of an uncharged oxime reactivator herein, as a racemate or in enantiopure forms, can be accomplished in multiple steps. The preferred route, which as later discussed can be more generalized, starts with the reaction of 1-methyl-3,5-dintro-2-pyridone identified as compound (1) with 1,4-dioxaspiro[4.5]decan-8-one identified as compound (2) in an annulation reaction thereby forming the protected ketal compound 3′-nitro-7′,8′-dihydro-5′H-spiro[1,3]dioxolane-2,6′-quinoline] identified as compound (3). As illustrated below, the reaction is preferably conducted in the presence of ammonia (NH₃) and methanol (MeOH) at elevated temperature (e.g., 55° C.) and resulted in a 79% yield.

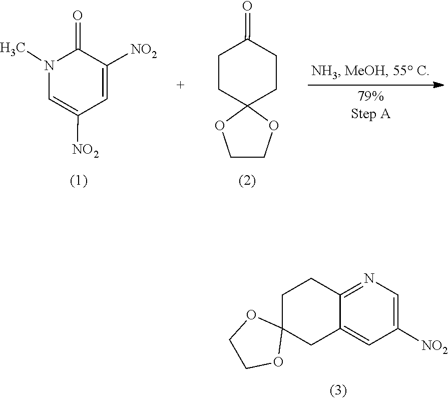 [SMILES: Cn1cc([N+](=O)[O-])cc([N+](=O)[O-])c1=O ; O=C1CCC2(CC1)OCCO2 ; O=[N+]([O-])c1cnc2c(c1)CC1(CC2)OCCO1]

The protected ketal compound (3) is then subject to hydrolysis preferably in the presence of sulfuric acid (20%) at room temperature (rt) to form 3-nitro-7,8-dihydroquinolin-6 (5H)-one identified as compound (4), as illustrated below:

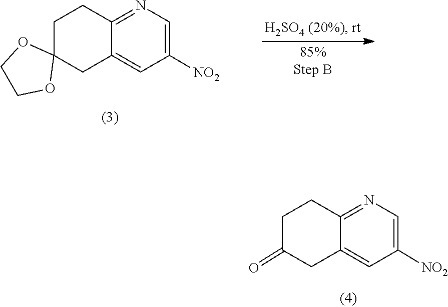 [SMILES: O=[N+]([O-])c1cnc2c(c1)CC1(CC2)OCCO1 ; O=C1CCc2ncc([N+](=O)[O-])cc2C1]

In general embodiment, compound 4 is then subject to reductive amination upon treatment with Ar—(R₁)—NH—R₂, wherein Ar can be an aromatic or heteroaromatic ring, R₁ is an alkyl group having 2-4 carbon atoms and R₂ is a saturated or unsaturated hydrocarbon group having 1-4 carbon atoms. In preferred embodiment, Ar is an aromatic group, R₁ is —CH₂CH₂— and R₂ is —CH₃, and one therefore utilizes N-methyl-2-phenylethane-1-amine preferably in the presence of titanium isopropoxide (Ti(OiPr)₄), sodium triacetoxyborohydride (NaHB(OAc)₃), acetic acid (AcOH), and 1,2-dichloroethane (DCE) at room temperature as illustrated below which forms the racemic intermediate (±)-N-methyl-3-nitro-N-phenethyl-5,6,7,8-tetrahydroquinolin-6-amine identified as (±)-(5).

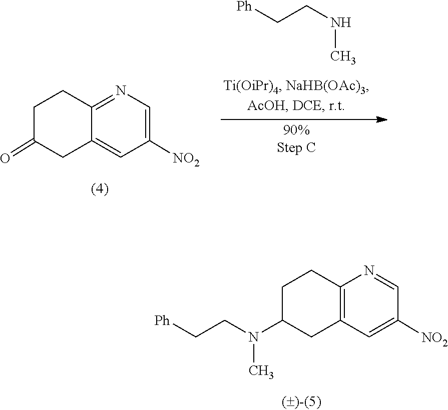

Reduction of the nitro moiety on compound (±)-(5) preferably in the presence of trichlorosilane (HSiCl₃), triethylamine (TEA) in acetonitrile (MeCN) at 0° C. forms (±)-N6-methyl-N6-phenethyl-5,6,7,8-tetrahydroquinoline-3,6-diamine identified as (±)-(6) at 84% yield.

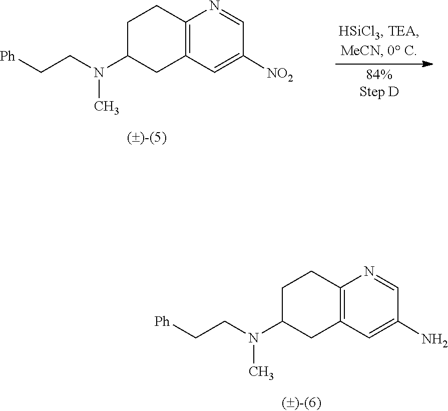 [SMILES: CN(CCc1ccccc1)C1CCc2ncc([N+](=O)[O-])cc2C1 ; CN(CCc1ccccc1)C1CCc2ncc(N)cc2C1]

Compound (±)-(6) is then preferably reactively coupled to the acid chloride (E)-2-((allyloxy)imino)-3-oxobutanoyl chloride identified as compound (7), preferably in the presence of N-methylmorpholine (NMM) in ethyl acetate (EtOAc) to form the allyl protected (±)-2-((allyloxy)imino)-N-(6-(methyl(phenethyl)amino)-5,6,7,8-tetrahydroquinolin-3-yl)-3-oxobutanamide identified as (±)-(8) which can undergo deprotection of the allyl group (e.g., conversion into an —OH group) preferably in the presence of tetrakis(triphenylphosphane) palladium (0) Pd(PPh₃)₄ and N-methylmorpholine (NMM), to form (±)-2-(hydroxyimino)-N-(6-(methyl(phenethyl)amino)-5,6,7,8-tetrahydroquinolin-3-yl)-3-oxobutanamide identified as (±)-(9). Reference to the allyl group herein is reference to the group —CH₂—CH═CH₂.

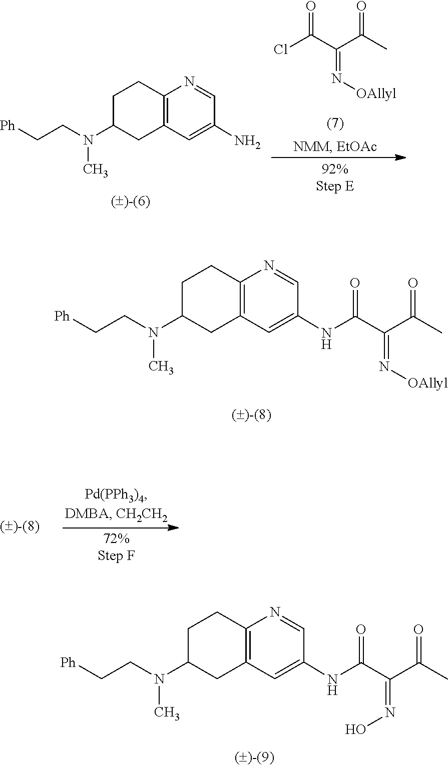

It is worth noting that with regards to the acid chloride coupling noted above, access to a requisite amount of (E)-2-((allyloxy)imino)-3-oxobutanoyl chloride, identified as compound (7), is desired. Preferably, as described below, this can be achieved by stockpiling an oxime salt fragment, 2-methylpropan-2-aminium (Z)-2-((allyloxy)imino)-3-oxobutanoate, identified as compound (14) as a relatively stable salt, and converting (14) as needed to form the acid chloride (7). Such preferred process begins with oxidation of tert-butyl-3-oxobutanoate identified as compound (10), in the presence of sodium nitrate (NaNO₂) in acetic acid (AcOH) to form (Z)-tert-butyl 2-(hydroxyimino)-3-oxobutanoate, identified as compound (11), at 84% yield.

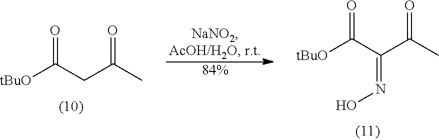

Alkylation of (11) with allyl bromide in the presence of potassium carbonate (K₂CO₃) in acetone formed (Z)-tert-butyl 2-((allyloxy)imino)-3-oxobutanoate identified as compound (12), at 94% yield and the tert butyl group is subsequently removed with in the presence of sulfuric acid and acetic acid to provide (Z)-tert-butyl 2-((allyloxy)imino)-3-oxobutanoate, identified as compound (13), at 94% yield.

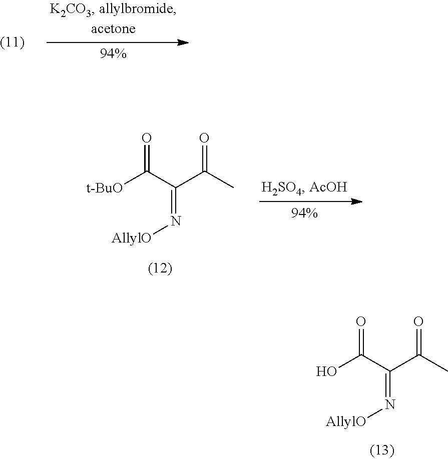

Compound (13) as the free acid was then subsequently crystallized via the tert-butyl amine salt to form 2-methylpropan-2-aminium (Z)-2-((allyloxy)imino)-3-oxobutanoate, identified as compound (14), which was observed to be stable for several months at room temperature with no change in purity. Anhydrous salt cracking of (14) with sulfuric acid in ethyl acetate (EtOAc) forms (13) followed by treatment with oxalyl chloride generated the acid chloride (7), which as noted above, can be used for coupling to compound (6) to form compound (8).

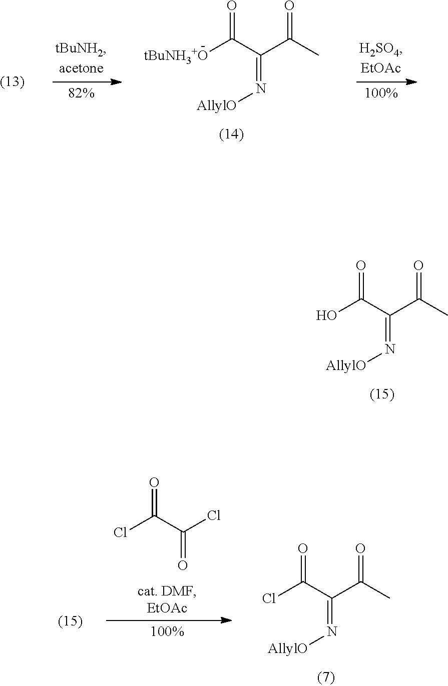

It should be appreciated from the above that one can therefore more generally prepare other amine salts having the following general formula, where R in such formula can be a hydrogen or an alkyl group, such as methyl, ethyl, butyl or in preferred embodiment, as illustrated above for compound 14, the t-butyl group (tBu).

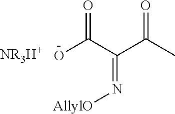

As alluded to herein, the present invention provides for the ability to isolate enantiopure compounds (+)-(9) or (−)-9. This is now achieved via a chiral resolution of the preferred racemic intermediate (±)-N-methyl-3-nitro-N-phenethyl-5,6,7,8-tetrahydroquinolin-6-amine identified as (±)-(5), preferably using dibenzoyl-D-tartaric acid and/or dibenzyl-L-tartaric acid as illustrated below.

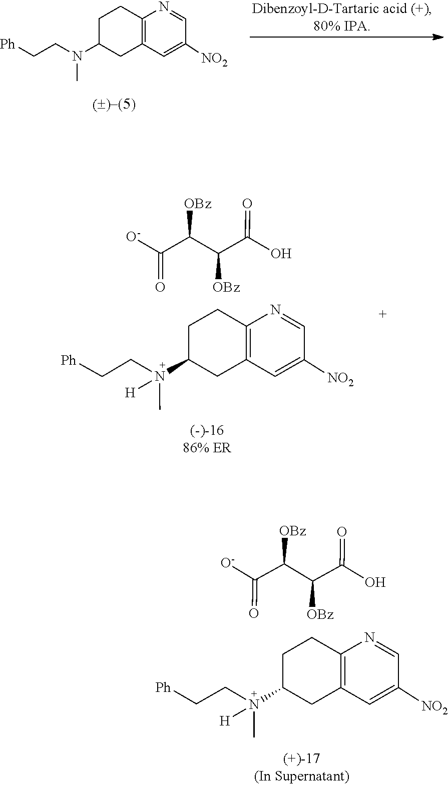

Accordingly, crystallization of (±)-(5) with dibenzoyl-D-tartaric acid produces compound (−)-16 with 86% enantiomeric ratio (ER) which can be subsequently recrystallized to achieved 99% ER in an overall yield of 38% from (±)-(5).

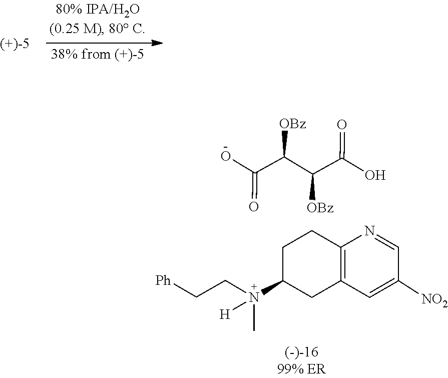

The supernatant, enriched in (+)-17 can then be concentrated, and the free base is liberated with either sodium hydroxide or potassium hydroxide and subsequently crystallized with dibenzoyl-L-tartaric acid to isolate intermediate (+)-N-methyl-3-nitro-N-phenethyl-5,6,7,8-tetrahydroquinolin-6-amine, identified as (±)-17 with 98% ER. After liberation of the free base, intermediates (−)-16 and (+)-17 can then be advanced through Steps D, E and F herein to preferably isolate (−)-2-(hydroxyimino)-N-(6-(methyl(phenethyl)amino)-5,6,7,8-tetrahydroquinolin-3-yl)-3-oxobutanamide identified as (−)-(9) or (+)-2-(hydroxyimino)-N-(6-(methyl(phenethyl)amino)-5,6,7,8-tetrahydroquinolin-3-yl)-3-oxobutanamide identified as (+)-(9).

Accordingly, in the broad context of the present disclosure, the free base form of (−)-(16) can therefore have the following formula:

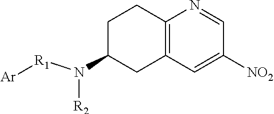

and the free base form of (+)-17 can therefore have the following formula:

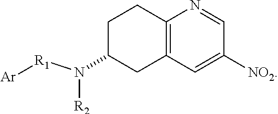

As may now be appreciated, via use of Ar—(R₁)—NH—R₂ in Step C, wherein Ar can be an aromatic or heteroaromatic ring, R₁ is an alkyl group having 2-4 carbon atoms and R₂ is a saturated or unsaturated hydrocarbon group having 1-4 carbon atoms, one can readily prepare the more general forms of compounds (5), (6) and (8) above, which can respectively be illustrated as follows:

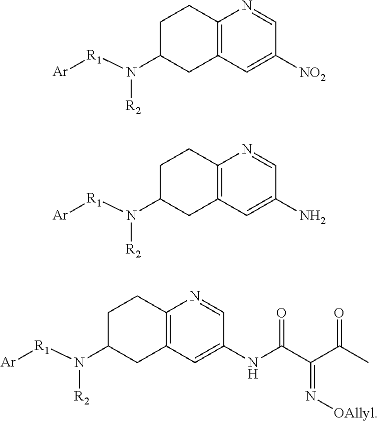

The following general reactivator structures, as a racemate, or in enantiopure form, can also now be produced herein. Again, this is reference to the feature that the desired enantiomer form of the structure below constitutes at least 90% of all forms of the compound, more preferably at least 95% of all forms of the compound, and most preferably, at least 98% or 99% of all forms of the compound.

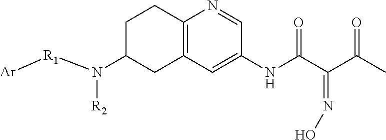

Once prepared, the uncharged oxime reactivators herein may be administered for treating an organophosphate toxicity or poisoning or toxic exposure, or for treating organophosphate inhibition of an acetylcholinesterase (AChE) by administering to a patient or individual in need thereof, a racemate having the following general formula:

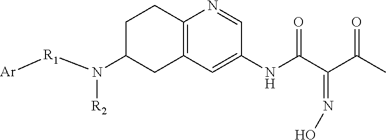

and/or the enantiopure forms herein having the following general formulas:

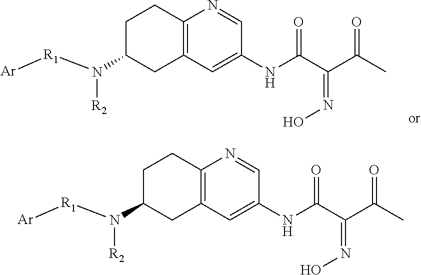

wherein again Ar can be an aromatic or heteroaromatic ring, R₁ is an alkyl group having 2-4 carbon atoms and R₂ is a saturated or unsaturated hydrocarbon group having 1-4 carbon atoms. The above racemate and/or enantiopure forms may also be provided in a pharmaceutically acceptable carrier.

The pharmaceutically acceptable carrier may therefore be understood herein as an aqueous formulation containing the above reference racemate enantiopure forms. Such formulations may therefore amount to aqueous solutions, suspensions and/or emulsions. Furthermore, it may be appreciated that an emulsion may be understood as an aqueous mixture containing the subject compounds in the presence of suitable hydrophobic/hydrophilic emulsifying agents.

Accordingly, the racemate or the enantiopure forms of

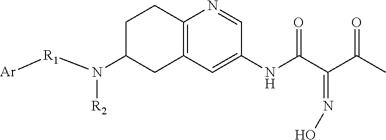

may preferably be present herein in an aqueous formulation at a concentration of 0.1-50% by weight, including all values and increments therein. It may be appreciated, however, that the specific doses may depend on a variety of factors, for example, the age, body weight, general state of health and time of administration and the time and severity of exposure. It is worth noting that parenteral administration may be utilized herein, whether for prophylaxis or therapeutically (i.e., before exposure to a cholinesterase inhibitor).

In addition, the pharmaceutically acceptable aqueous carrier herein may include other diluents suitable for preparing oral pharmaceutical suspension. For example, an oral pharmaceutical suspension of the present invention may include, if necessary, pharmaceutically acceptable additives including auxiliary substances, stabilizing agents, suspending agents, surface tension modifiers, viscosity modifiers, colorants, preservatives, flavoring agents and other commonly used additives such as lactose, citric acid, tartaric acid, stearic acid, magnesium stearate, sucrose, and the like.

1. A method for producing an oxime reactivator comprising:
(a) reacting 3,5-dinitro-2 (1H)-pyridone with 1,4-dioxaspiro[4.5]decan-8-one and forming the 3′-nitro-7′,8′-dihydro-5′H-spiro dioxolane-2,6′-quinoline] having the following formula: 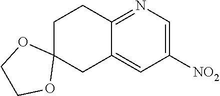 [SMILES: O=[N+]([O-])c1cnc2c(c1)CC1(CC2)OCCO1]
(b) hydrolyzing 3′-nitro-7′,8′-dihydro-5′H-spiro[1,3]dioxolane-2,6′-quinoline] and forming 3-nitro-7,8-dihydroquinolin-6 (5H)-one having the following formula: 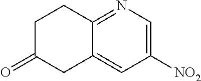 [SMILES: O=C1CCc2ncc([N+](=O)[O-])cc2C1]
(c) subjecting 3-nitro-7,8-dihydroquinolin-6 (5H)-one to reductive amination with Ar—(R₁)—NH—R₂, wherein Ar can be an aromatic or heteroaromatic ring, R₁ is an alkyl group having 2-4 carbon atoms and R₂ is a saturated or unsaturated hydrocarbon group having 1-4 carbon atoms and producing 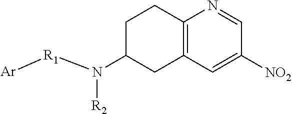
(d) reducing the nitro group on the compound formed in (c) and forming: 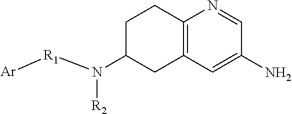
(e) coupling the compound formed in (d) with (E)-2-((allyloxy)imino)-3-oxobutanoyl chloride to form: 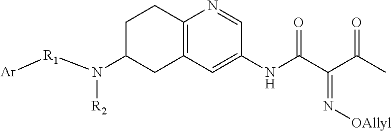
(f) conversion of the allyl group in the compound produced in (e) and forming:

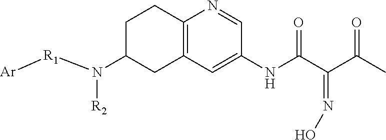

2. The method of claim 1 wherein the compound formed in in step (c) undergoes chiral resolution to form 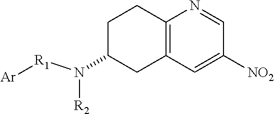
which is reduced in step (d), coupled in step (e) and converted in step (f) to recover the following: 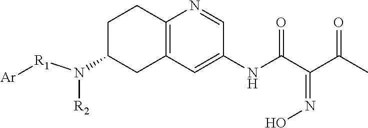
at an enantiomeric purity of at least 90%.

3. The method of claim 1 wherein the enantiomeric purity is at least 95%.

4. The method of claim 3 wherein the enantiomeric purity is at least 98%.

5. The method of claim 3 wherein the enantiomeric purity is at least 99%.

6. The method of claim 1 wherein in the compound in step (c) undergoes chiral resolution to form: 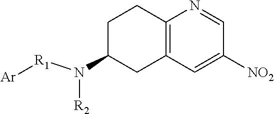
which is reduced in step (d), coupled in step (e) and converted in step (f) to recover the following: 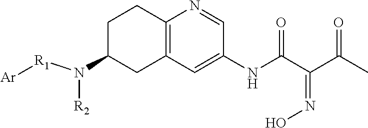
at an enantiomeric purity of at least 90%.

7. The method of claim 6 wherein the enantiomeric purity is at least 95%.

8. The method of claim 6 wherein the enantiomeric purity is at least 98%.

9. The method of claim 6 wherein the enantiomeric purity is at least 99%.

10. The method of claim 1 wherein said (E)-2-((allyloxy)imino)-3-oxobutanoyl chloride is derived from the following amine salt: 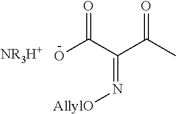
where R is a hydrogen or an alkyl group.

11. The method of claim 1 wherein said reductive amination in step (c) is carried out with Ar—(R₁)—NH—R₂ wherein Ar is a benzene ring, R₁ is —CH₂CH₂— and R₂ is —CH₃ and forming in step (f):

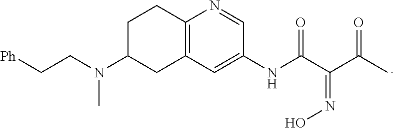 [SMILES: CC(=O)/C(=N/O)C(=O)Nc1cnc2c(c1)CC(N(C)CCc1ccccc1)CC2]

12. The method of claim 2 wherein the compound recovered in step (f) comprises:

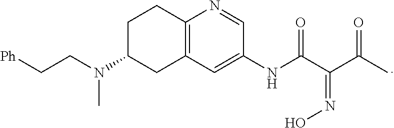 [SMILES: CC(=O)/C(=N/O)C(=O)Nc1cnc2c(c1)C[C@H](N(C)CCc1ccccc1)CC2]

13. The method of claim 6 wherein the compound recovered in step (f) comprises:

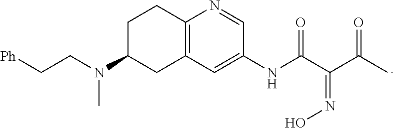 [SMILES: CC(=O)/C(=N/O)C(=O)Nc1cnc2c(c1)C[C@@H](N(C)CCc1ccccc1)CC2]

14. A method of reactively coupling with an acid chloride comprising:
(a) providing the following compound having the formula: 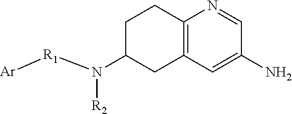
wherein Ar can be an aromatic or heteroaromatic ring, R₁ is an alkyl group having 2-4 carbon atoms and R₂ is a saturated or unsaturated hydrocarbon group having 1-4 carbon atoms;
(b) providing an acid chloride compound having the following formula wherein Allyl is reference to —CH₂—CH═CH₂: 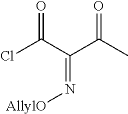
(c) coupling the compounds in (a) and (b) and forming:

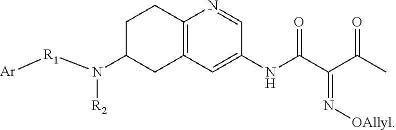

15. An enantiopure compound comprising the following formula: 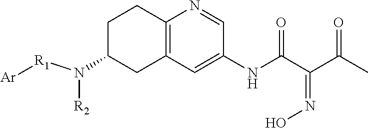
wherein Ar can be an aromatic or heteroaromatic ring, R₁ is an alkyl group having 2-4 carbon atoms, R₂ is a saturated or unsaturated hydrocarbon group having 1-4 carbon atoms and having an enantiomeric purity of at least 90%.

16. The enantiopure compound of claim 15 having an enantiomeric purity of at least 95%.

17. The enantiopure compound of claim 15 having an enantiomeric purity of at least 98%.

18. The enantiopure compound of claim 15 having an enantiomeric purity of at least 99%.

19. An enantiopure compound comprising the following formula: 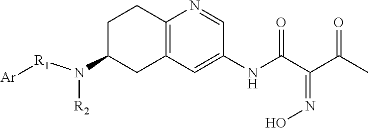
wherein Ar can be an aromatic or heteroaromatic ring, R₁ is an alkyl group having 2-4 carbon atoms, R₂ is a saturated or unsaturated hydrocarbon group having 1-4 carbon atoms and having an enantiomeric purity of at least 90%.

20. The enantiopure compound of claim 19 having an enantiomeric purity of at least 95%.

21. The enantiopure compound of claim 19 having an enantiomeric purity of at least 98%.

22. The enantiopure compound of claim 19 having an enantiomeric purity of at least 99%.

23. A therapeutic method of treating a person or animal for intoxication with a phosphorous containing cholinesterase inhibitor, comprising administering to a person or animal one or more of the following compounds:
(a) an enantiopure compound comprising the following formula: 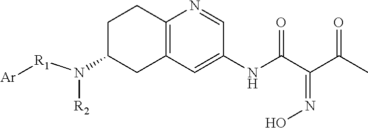
wherein Ar can be an aromatic or heteroaromatic ring, R₁ is an alkyl group having 2-4 carbon atoms, R₂ is a saturated or unsaturated hydrocarbon group having 1-4 carbon atoms and having an enantiomeric purity of at least 90%; or
(b) an enantiopure compound having the following formula: 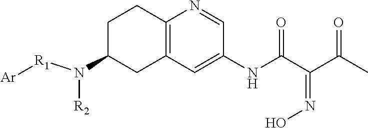
wherein Ar can be an aromatic or heteroaromatic ring, R₁ is an alkyl group having 2-4 carbon atoms, R₂ is a saturated or unsaturated hydrocarbon group having 1-4 carbon atoms and having an enantiomeric purity of at least 90%.
